(19) United States
(12) Patent Application Publication
(10) Pub. No.: US 2014/0319374 A1
(43) Pub. Date: Oct. 30, 2014

(54) Portable Ultraviolet Pulse Oximeter Sanitizer

(71) Applicant: David Chandler, Rougemont, NC (US)

(72) Inventor: David Chandler, Rougemont, NC (US)

(21) Appl. No.: 13/871,860

(22) Filed: Apr. 26, 2013

(51) Int. Cl. A61L 2/10

(52) U.S. Cl. CPC A61L 2/10
USPC 250/455.11

(57) ABSTRACT

A portable ultraviolet pulse oximeter sanitizer is a handheld unit that irradiates a pulse oximeter probe with ultraviolet light within a sealed sanitizing chamber. A main compartment section has one half of the sanitizing chamber, and a door section has the other half of the sanitizing chamber. A pulse oximeter probe may be clamped onto an attachment post within the sanitizing chamber. After the door is closed, a plurality of ultraviolet lamps within the sanitizing chamber are activated, irradiating the pulse oximeter probe with ultraviolet light. A battery provides electrical power to the ultraviolet lamps, and a timer automatically deactivates the ultraviolet lamps after a specified time period.

### FIELD OF THE INVENTION

The present invention relates generally to medical equipment. More specifically, the present invention relates to sanitization of pulse oximetry equipment.

### BACKGROUND OF THE INVENTION

Hemoglobin is an iron-containing oxygen-transport metalloprotein in the red blood cells of nearly all vertebrate animals, as well as in the tissue of some invertebrates. Hemoglobin in the blood is responsible for carrying oxygen from the respiratory organs (lungs or gills) to the rest of the body, where it releases the oxygen to burn nutrients to provide energy for powering the various functions of the organism. Hemoglobin also collects the resultant carbon dioxide and brings it back to the respiratory organs to be dispensed from the organism. Hemoglobin is also involved in the transport of other gases such as the important regulatory molecule nitric oxide, and has other functions such as functioning as an antioxidant and a regulator of iron metabolism in various tissues. Hemoglobin concentration measurement is among the most commonly performed blood tests. Hemoglobin deficiency is associated with various disease symptoms such as anemia, nutritional deficiency, bone marrow problems, and kidney failure.

Pulse oximetry is a non-invasive method that allows the monitoring of the saturation of a patient's hemoglobin. A sensor is placed on a thin part of the patient's body, usually a fingertip or earlobe. Light of two different wavelengths is passed through the patient to a photodetector. The changing absorption of light radiation as a function of the frequency or wavelength due to its interaction with a sample is measured for each of the wavelengths. This allows determination of the absorbances due to the pulsing arterial blood alone, excluding venous blood, skin, bone, muscle, and fat. Absorption of certain wavelengths of light differs significantly between oxyhemoglobin and its deoxygenated form, allowing the oxy/deoxyhemoglobin ratio to be calculated from the ratio of the absorption of the two wavelengths of light. This ratio is generally indicative of the oxygen saturation of a patient's blood. The need for oxygen is the most essential element for life, and assessing a patient's need for oxygen is often invaluable in diagnosing the patient's condition in order to provide appropriate treatment.

A pulse oximeter is useful in any setting where a patient's oxygenation is unstable, including intensive care, operating, recovery, emergency and hospital ward settings, pilots in unpressurized aircraft, assessment of any patient's oxygenation, and determining the effectiveness or need for supplemental oxygen. Pulse oximeters are very simple and easy to use (the alternative being having a blood sample tested in a lab) and are capable of providing continuous and immediate oxygen saturation values, making them of critical importance in emergency medicine. Pulse oximeters are also very useful for patients with respiratory or cardiac problems, especially chronic obstructive pulmonary disease (COPD), or for diagnosis of some sleep disorders such as apnea and hypo apnea. Portable pulse oximeters are also useful for mountain climbers and other athletes whose oxygen levels may decrease at high altitudes or with exercise.

As with any medicinally related equipment and practices, pulse oximeters should be held to a standard for cleanliness and sanitization. Sterilization is a term referring to any process that eliminates or kills all forms or the vast majority of microbial life, including transmissible agents such as fungi, bacteria, viruses, or spore forms present on a surface, contained in a fluid, in medication, or in a compound such as biological culture media. Sterilization can be achieved by applying the proper combinations of heat, chemicals, irradiation, high pressure, and filtration. However, sterilization is difficult to achieve and often impractical. A related process known as sanitization is less effective than sterilization, but more practical as the effort or equipment required for complete sterilization may not be worth the result. Sanitization does not get rid of 100% of microbial life, but generally is able to get close, killing around 99.9% of microbial life, depending on the method used. Pulse oximeters are typically not disposed of after a single use, and therefore must be disinfected between uses to prevent transmission of harmful microbial agents from patient to patient. Currently, pulse oximeters are generally disinfected simply by wiping exposed surfaces with a cloth or pad moistened with a detergent solution, medical alcohol or another disinfectant. This may be less than fully effective as a person may miss spots due to careless cleaning, and is also inconvenient to do after every use of a pulse oximeter.

Ultraviolet germicidal irradiation (UVGI) is a disinfection method that utilizes ultraviolet (UV) light at sufficiently short wavelength to kill microorganisms. UVGI is effective in destroying the nucleic acids in these organisms so that their deoxyribonucleic acid (DNA) is disrupted by the UV radiation, leaving them unable to perform vital cellular functions. The wavelength of UV that causes this effect is rare on Earth as the atmosphere blocks it. UV has primarily been employed in medical sanitation, sterile work facilities and increasingly to sterilize drinking water and wastewater. A portable unit that utilizes UVGI to sanitize pulse oximeters would allow medical personnel to conveniently and efficiently maintain higher sanitation standards than currently employed with regard to pulse oximeters.

It is therefore an object of the present invention to provide a portable pulse oximeter sanitizer that utilizes UVGI to sanitize pulse oximeter units.

### BRIEF DESCRIPTION OF THE DRAWINGS

FIG. 1 is a right perspective view of the present invention with the door compartment section in the closed position.

FIG. 2 is a front elevation view of the present invention with the door compartment section in the open position.

FIG. 3 is a right perspective view of the present invention with the door compartment section in the open position.

FIG. 4 is a top plan view of the present invention with the door compartment section in the closed position.

FIG. 5 is a lower left perspective view of the present invention with the door compartment section in the open position and with the battery door open.

FIG. 6 is schematic showing the electrical connections of a simple form of the present invention.

### DETAIL DESCRIPTIONS OF THE INVENTION

All illustrations of the drawings are for the purpose of describing selected versions of the present invention and are not intended to limit the scope of the present invention.

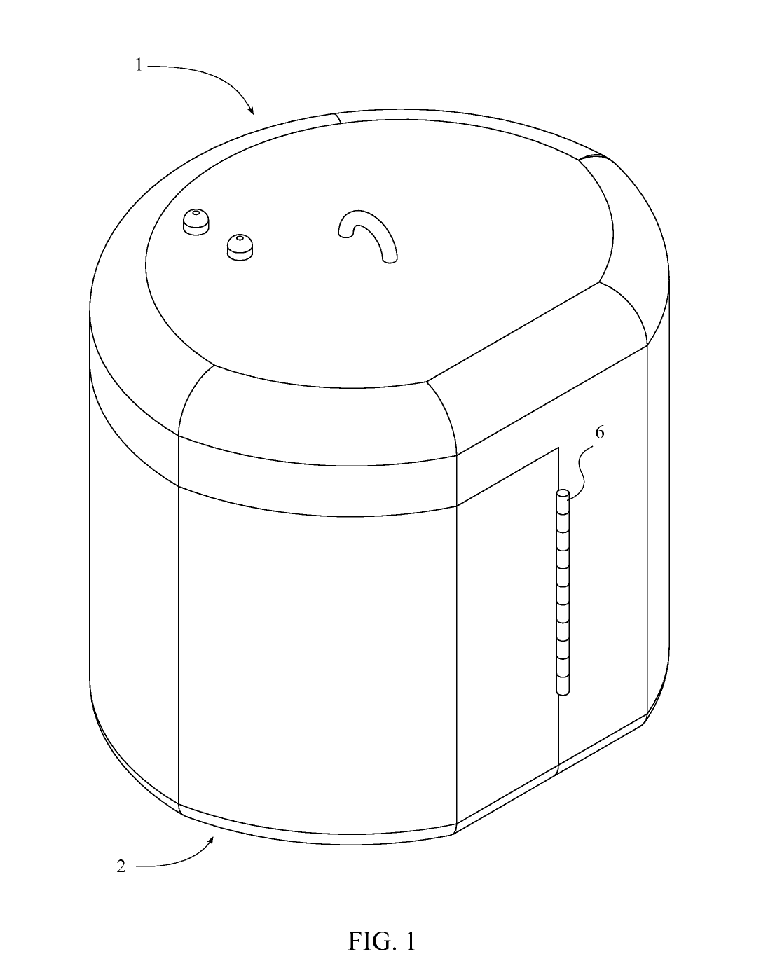

The present invention is a portable, handheld, standalone device that provides the capability to medical professionals to quickly, conveniently and effectively sanitize pulse oximeter units using ultraviolet germicidal irradiation (UVGI). Referring to FIGS. 1-3, a door opens and closes to reveal or seal a chamber within a main compartment. A pulse oximeter probe is clamped onto a post within the chamber and the door is closed. Closing the chamber completes an electrical circuit between electrical connectors on the main compartment and the door, initiating a timer and activating ultraviolet (UV) lamps positioned within the chamber, exposing the pulse oximeter probe to sanitizing ultraviolet light for a specified time interval. The connectors on the main compartment and the door also act as a failsafe feature, preventing exposure of the user to ultraviolet light. In the preferred embodiment, the present invention is lightweight and disposable, being manufactured with a built-in battery and the structural components being made substantially from lightweight plastic. In alternate embodiments, the battery is replaceable or the present invention is made from other materials or additional materials, including, but not limited to, more heavy-duty plastic, rubber, metal, or any other appropriate materials.

The present invention generally comprises a main compartment section 1, a door compartment section 2, a sanitizing chamber 3, a plurality of ultraviolet lamps 4, at least one battery 5, a hinge 6, and a timer 7. The main compartment section 1 is substantially cylindrical and the bulk of the present invention, and comprises a battery chamber 11, a lanyard attachment eyelet 13, and a plurality of female electrical sockets 14. The lanyard attachment eyelet 13 is a hook, ring, clasp or any other physical structure that allows the present invention to be easily attached to a lanyard and carried thereby. The lanyard attachment eyelet 13 is preferably positioned atop the main compartment section 1 opposite the sanitizing chamber 3, but may be positioned in any other location in alternate embodiments.

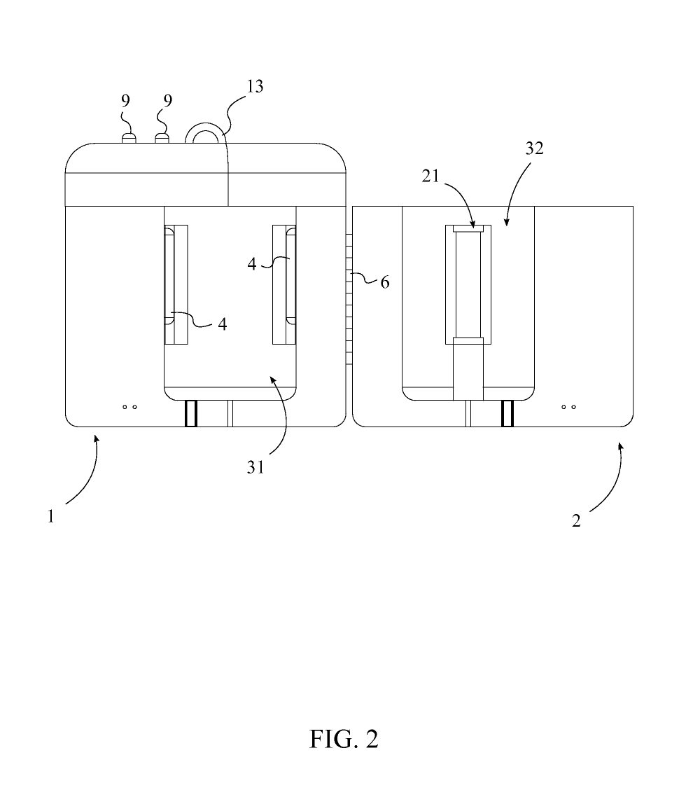

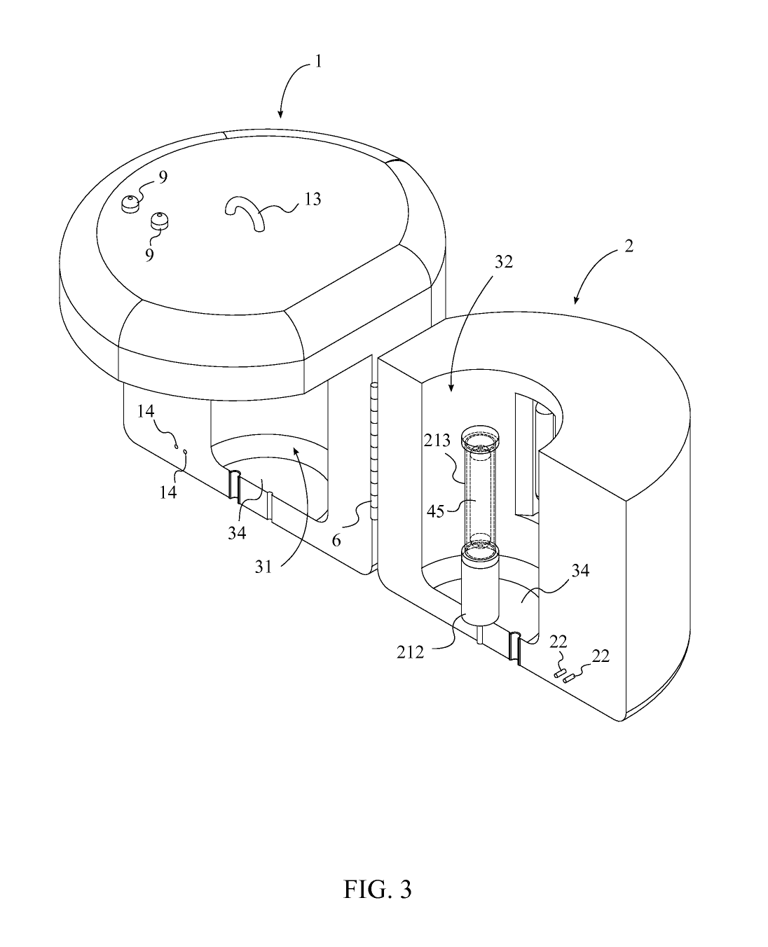

As can be seen in FIGS. 2-3, the door compartment section 2 comprises an oximeter attachment post 21 and a plurality of male electrical probes 22, and is hingedly connected to the main component section by the hinge 6. The hinge 6 is oriented vertically, so that the door compartment section 2 swings open and closed by rotating horizontally about the hinge 6. The main compartment section 1 and the door compartment section 2 preferably comprise any commonly known elements that function to fix the door compartment section 2 in the closed position when in use, such as, but not limited to, a latch, clasp, or button snaps.

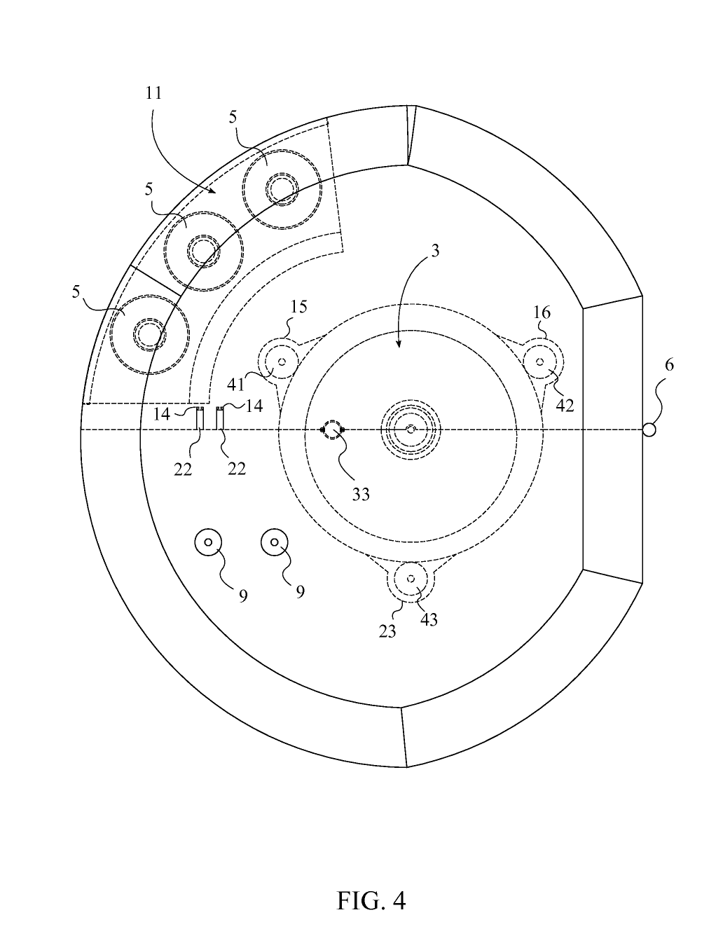

Referring to FIGS. 3-4, the sanitizing chamber 3 traverses vertically through portions of both the main compartment section 1 and the door compartment section 2. The sanitizing chamber 3 is an enclosed space within the present invention that is accessed by opening the door compartment section 2. The interior surfaces of the sanitizing chamber 3 are preferably made with or covered by aluminum for its reflective properties, so that UV rays are reflected around the interior of the sanitizing chamber, facilitating more effective coverage of every surface within the sanitizing chamber 3 with UV radiation. A pulse oximeter probe is placed within the chamber for sanitization. The plurality of ultraviolet lamps 4 is used to sanitize the pulse oximeter probe using UVGI. The plurality of ultraviolet lamps 4 is within the sanitizing chamber 3 or adjacent to the sanitizing chamber 3 separated by a pane of translucent material, and are positioned around the sanitizing chamber 3 in order to maximize exposure of all the surfaces of the pulse oximeter probe to the UV radiation generated by the plurality of ultraviolet lamps 4.

In the preferred embodiment of the present invention, the sanitizing chamber 3 comprises a main sanitizing chamber portion 31 and a door sanitizing chamber portion 32. Preferably, the main sanitizing chamber portion 31 and the door sanitizing chamber portion 32 are each half of the sanitizing chamber 3, though the main sanitizing chamber portion 31 and the door sanitizing chamber portion 32 may be more or less than half of the sanitizing chamber 3. Preferably, the sanitizing chamber 3 is cylindrical, though it is understood that in alternate embodiments the sanitizing chamber 3 may take any geometric shape that is conducive to the spirit of the present invention. The main sanitizing chamber portion 31 is positioned within the main compartment section 1, and the door sanitizing chamber portion 32 is positioned within the door compartment section 2.

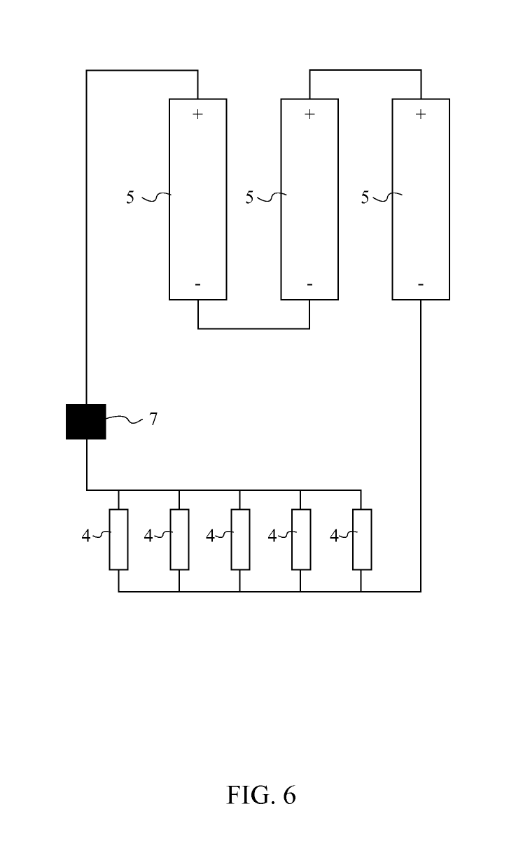

When the door compartment section 2 is in a closed position relative to the main compartment section 1, the main sanitizing chamber portion 31 and the door sanitizing chamber portion 32 are positioned concentrically with and adjacent to each other, and the sanitizing chamber 3 is thus sealed, preventing any UV radiation from escaping the sanitizing chamber 3. The plurality of female electrical sockets 14 and the plurality of male electrical probes 22 complete an electrical circuit, initiating a timing circuit when connected and breaking the electrical circuit by being separated when the door compartment section 2 is not in the closed position. Referring to FIG. 6, the electrical circuit is a series of electrical components that are electrically connected to each other in series or in parallel when the door compartment section 2 is in the closed position. It should be noted that the plurality of female electrical sockets 14 and the plurality of male electrical probes 22 are not limited to being female electrical sockets and male electrical probes, respectively, but may be any type of electrical connectors which complete an electrical circuit by contact with each other when the door compartment section 2 is in the closed position. In an alternate embodiment of the present invention, the sanitizing chamber 3 is positioned substantially or entirely within the main compartment section 1, and the door compartment section 2 functions only to allow access to the sanitizing chamber 3.

In the preferred embodiment of the present invention, the sanitizing chamber 3 further comprises a probe cable channel 33. The probe cable channel 33 traverses through the walls of the sanitizing chamber 3 to the outside environment, preferably vertically downward through the floor 34 of the sanitizing chamber 3. The probe cable channel 33 is preferably positioned laterally offset from the center of the sanitizing chamber 3. The probe cable channel 33 is necessary since many pulse oximeter probes are permanently attached to a control unit via a probe cable; thus, a channel must be provided to accommodate the probe cable and allow the pulse oximeter probe to be positioned within the sanitizing chamber 3 while simultaneously allowing the probe cable to traverse from the sanitizing chamber 3 to the outside environment when the door compartment section 2 is in the closed position. The probe cable channel 33 is preferably lined with an elastic or compressible material such as rubber or foam in order to effectively seal the probe cable channel 33 against any ultraviolet light escaping from the sanitizing chamber 3 when the present invention is in use. An alternate embodiment of the present invention does not comprise the probe cable channel 33 to accommodate pulse oximeter probes that may be disconnected from the probe cable.

In the preferred embodiment of the present invention, the oximeter attachment post 21 is oriented vertically so that a pulse oximeter probe may be clamped onto the oximeter attachment post 21. When the door compartment section 2 is closed, the oximeter attachment post 21 and the pulse oximeter probe are positioned within the sanitizing chamber 3. The oximeter attachment post 21 is concentrically positioned within the sanitizing chamber 3 when the door compartment section 2 is in the closed position. In the preferred embodiment of the present invention, the oximeter attachment post 21 is concentrically positioned within the door sanitizing chamber portion 32, though in an alternate embodiment the oximeter attachment post 21 is concentrically positioned within the main sanitizing chamber 3 portion.

Referring to FIG. 3, the oximeter attachment post 21 comprises a post base 212, a post lamp chamber 213 and a post ultraviolet lamp 45. In the preferred embodiment of the present invention, the post base 212 is connected to a floor 34 of the sanitizing chamber 3. The post base 212 and the post lamp chamber 213 are concentrically positioned with each other. The post lamp chamber 213 is connected atop the post base 212 opposite the floor 34. The post ultraviolet lamp 45 is positioned within the post lamp chamber 213. The post lamp chamber 213 is translucent so that the post ultraviolet lamp 45 may irradiate the inside surfaces of the pulse oximeter probe that are clamped onto the oximeter attachment post 21. The post ultraviolet lamp 45 is electrically connected to the at least one battery 5, and the timer 7.

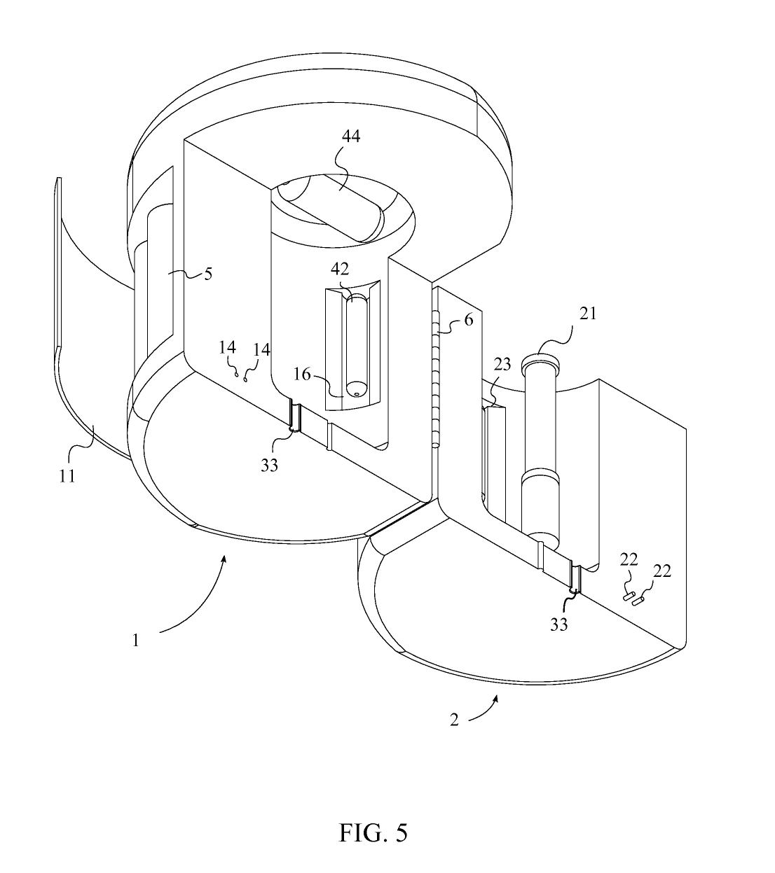

Referring to FIGS. 3-6, in the preferred embodiment of the present invention, the plurality of ultraviolet lamps 4 comprises a first ultraviolet lamp 41, a second ultraviolet lamp 42, a third ultraviolet lamp 43, and an upper ultraviolet lamp 44. The post ultraviolet lamp 45 may also be known as belonging to the plurality of ultraviolet lamps 4. The first ultraviolet lamp 41, the second ultraviolet lamp 42, and the third ultraviolet lamp 43 are oriented parallel to each other. The first ultraviolet lamp 41, the second ultraviolet lamp 42, and the third ultraviolet lamp 43 are perimetrically positioned around the sanitizing chamber 3. The first ultraviolet lamp 41, the second ultraviolet lamp 42, and the third ultraviolet lamp 43 are equally spaced apart from each other around the sanitizing chamber 3. The upper ultraviolet lamp 44 is centrally positioned within the sanitizing chamber 3 above the oximeter attachment post 21. The first ultraviolet lamp 41, the second ultraviolet lamp 42, and the third ultraviolet lamp 43 are positioned within a first lamp cavity 15, a second lamp cavity 16, and a third lamp cavity 23, respectively. Each of the first lamp cavity 15, second lamp cavity 16, and third lamp cavity 23 are sections cut into the walls of the sanitizing chamber 3 to accommodate the first ultraviolet lamp 41, the second ultraviolet lamp 42, and the third ultraviolet lamp 43. Alternate embodiments of the present invention may comprise additional or fewer ultraviolet lamps in different arrangements. It should be understood that the number and arrangement of the plurality of ultraviolet lamps 4 is secondary to the core function of the present invention, which is to expose all surfaces of the pulse oximeter probe as thoroughly as possible with UV radiation.

Referring to FIG. 4, the battery chamber 11 is positioned within the main compartment section 1 separately from the sanitizing chamber 3. For example, the battery chamber 11 may be positioned adjacent to the perimeter of the main compartment section 1. The at least one battery 5 is positioned within the battery chamber 11. The at least one battery 5 is electrically connected to the plurality of ultraviolet lamps 4, the timer 7, the plurality of female electrical sockets 14, and the plurality of male electrical probes 22. In the preferred embodiment of the present invention, the battery chamber 11 is not readily accessible by a user. In an alternate embodiment of the present invention, the main compartment section 1 further comprises a battery door 12 that is hingedly connected to the main compartment section 1 adjacent to the battery chamber 11. The battery door 12 allows a user access to the battery chamber 11 in order to replace the at least one battery 5 if the at least one battery 5 becomes drained from extended use. In another alternate embodiment, the present invention is not powered by a battery, but rather by a cord that may be plugged in to a typical 120 volt, 15 amp electrical wall socket, or other electrical wall sockets or power sources.

As can be seen in FIGS. 3-5, the plurality of male electrical probes 22 and the plurality of female electrical sockets 14 provide a failsafe connection for the entire electrical circuit. When the door compartment section 2 is in the closed position, the plurality of male electrical probes 22 contacts and is enveloped by the plurality of female electrical sockets 14, making an electrical connection between the plurality of male electrical probes 22 and the plurality of female electrical sockets 14. When the door compartment is open, the plurality of male electrical probes 22 and the plurality of female electrical sockets 14 are separated and therefore not electrically connected. The electrical circuit connecting the at least one battery 5 and the plurality of ultraviolet lamps 4 is contingent on the connection between the plurality of male electrical probes 22 and the plurality of female electrical sockets 14 in order to act as a failsafe, so that if a user opens the door compartment 2 the electrical circuit is instantly broken and the plurality of ultraviolet lamps 4 does not irradiate the user.

Referring to FIG. 6, in the preferred embodiment of the present invention, the timer 7 is a microchip or microprocessor that is electrically connected between the at least one battery 5 and the rest of the electrical circuit, specifically the plurality of ultraviolet lamps 4. In the preferred embodiment of the present invention, the plurality of ultraviolet lamps 4 is activated by closing the door compartment section 2 and making the electrical connection between the plurality of male electrical probes 22 and the plurality of female electrical sockets 14. After the electrical circuit is completed by closing the door compartment section 2, the timer 7 initializes and activated the plurality of ultraviolet lamps 4. After a specified time interval, such as, but not limited to, 10 seconds, the timer 7 deactivates the plurality of ultraviolet lamps by breaking the electrical connection between the at least one battery 5 and the plurality of ultraviolet lamps 4. In an alternate embodiment, the timer 7 uses analog means to control the duration of time the plurality of ultraviolet lamps 4 is activated.

In an alternate embodiment of the present invention, an external control interface allows a user to specify parameters relating to the exposure of the sanitizing chamber 3 to UV radiation, including, but not limited to, intensity, frequency, and duration of irradiation. The external control interface may be digital or analog. The preferred embodiment additionally includes one or more indicator lights 9 on the external surface of the main compartment section 1, the door compartment section 2, or both, indicating the current status of the irradiation process. For example, a red indicator light shows a user that the plurality of ultraviolet lamps 4 is currently activated, while a green indicator light reveals that the UV irradiation cycle has been completed and the door compartment section 2 may be opened to remove the pulse oximeter probe. Having the indicator lights 9 necessitates the electrical connection of the indicator lights 9 to the at least one battery 5 and the timer 7.

Although the invention has been explained in relation to its preferred embodiment, it is to be understood that many other possible modifications and variations can be made without departing from the spirit and scope of the invention as hereinafter claimed.

What is claimed is:

1. A portable ultraviolet pulse oximeter sanitizer comprises:
a main compartment section;
a door compartment section;
a sanitizing chamber;
a plurality of ultraviolet lamps;
at least one battery;
a hinge;
a timer;
the main compartment section comprises a battery chamber;
the door compartment section comprises an oximeter attachment post;
the door compartment section being hingedly connected to the main compartment section by the hinge, wherein the door compartment section opens and closes by rotating horizontally about the hinge;
the sanitizing chamber traversing through the main compartment section and the door compartment section;
the oximeter attachment post being concentrically positioned within the sanitizing chamber when the door compartment section is in a closed position;
the oximeter attachment post comprises a post ultraviolet lamp;

2. The portable ultraviolet pulse oximeter sanitizer as claimed in claim 1 comprises:
the sanitizing chamber comprises a main sanitizing chamber portion, a door sanitizing chamber portion, and a probe cable aperture, wherein the main sanitizing chamber portion and the door sanitizing chamber portion are each halves of the sanitizing chamber;
the main sanitizing chamber portion being positioned within the main compartment section;
the door sanitizing chamber portion being positioned within the door compartment section;
the main sanitizing chamber portion and the door sanitizing chamber portion being positioned concentric with and adjacent to each other when the door compartment section is in the closed position;
the oximeter attachment post being concentrically positioned within the door sanitizing chamber portion;

3. The portable ultraviolet pulse oximeter sanitizer as claimed in claim 1 comprises:
the oximeter attachment post further comprises a post base and a post lamp chamber, wherein the post lamp chamber is translucent;
the post base and the post lamp chamber being concentrically positioned with each other;
the post lamp chamber being connected atop the post base;
the post ultraviolet lamp being positioned within the post lamp chamber;
the post ultraviolet lamp being electrically connected to the at least one battery, and the timer;

4. The portable ultraviolet pulse oximeter sanitizer as claimed in claim 1 comprises:
the plurality of ultraviolet lamps comprises a first ultraviolet lamp, a second ultraviolet lamp, a third ultraviolet lamp, and an upper ultraviolet lamp;
the first ultraviolet lamp, the second ultraviolet lamp, and the third ultraviolet lamp being oriented parallel to each other;
the first ultraviolet lamp, the second ultraviolet lamp, and the third ultraviolet lamp being perimetrically positioned around the sanitizing chamber;
the first ultraviolet lamp, the second ultraviolet lamp, and the third ultraviolet lamp being equally spaced apart from each other around the sanitizing chamber;
the upper ultraviolet lamp being centrally positioned within the sanitizing chamber above the oximeter attachment post;

5. The portable ultraviolet pulse oximeter sanitizer as claimed in claim 1 comprises:
the battery chamber being positioned within the main compartment, wherein the battery chamber is positioned adjacent to the perimeter of the main compartment;
the at least one battery being positioned within the battery chamber;
the at least one battery being electrically connected to the plurality of ultraviolet lamps, and the timer;

6. The portable ultraviolet pulse oximeter sanitizer as claimed in claim 5 comprises:
the main compartment section further comprises a battery door;
the battery door being hingedly connected to the main compartment section adjacent to the battery chamber, wherein the battery door allows a user access to the at least one battery;

7. The portable ultraviolet pulse oximeter sanitizer as claimed in claim 1 comprises:
the main compartment comprises a lanyard attachment eyelet;
the lanyard attachment eyelet being positioned atop the main compartment section opposite the sanitizing chamber;

8. The portable ultraviolet pulse oximeter sanitizer as claimed in claim 1 comprises:
the main compartment section further comprises a plurality of female electrical sockets;
the door compartment section further comprises a plurality of male electrical probes;
the plurality of male electrical probes and the plurality of female electrical sockets being electrically connected to each other when the door compartment section is in the closed position;
the plurality of female electrical sockets being electrically connected to the at least one battery, wherein electrical power may only be delivered to the plurality of ultraviolet lamps when the plurality of female electrical sockets is electrically connected to the plurality of male electrical probes;
